(19) United States
(12) Patent Application Publication
(10) Pub. No.: US 2006/0190026 A1
(43) Pub. Date: Aug. 24, 2006

(54) Adjustable acupressure device

(76) Inventor: Laurie Sanders, Davie, FL (US)

Correspondence Address: MALLOY & MALLOY, P.A., 2800 S.W. Third Avenue, Historic Coral Way, Miami, FL, 33129, US

(21) Appl. No.: 11/065,400

(22) Filed: Feb. 24, 2005

(51) Int. Cl. A61B 17/00

(52) U.S. Cl. 606/204

(57) ABSTRACT

An adjustable acupressure device is structured to apply pressure to a specific portion of a user's body, for example, the hegu point located between the user's thumb and forefinger. The device includes an attachment assembly having at least one placement indicator structured to permit accurate placement of the device by the user on his or her body. Further, the device includes a pressure assembly to allow the user to apply a preselected amount of pressure to the specific portion of the user's body. More in particular, the pressure assembly includes a pressure applicator structured to contact the specific point on the user's body when disposed in an operative position.

### BACKGROUND OF THE INVENTION

1. Field of the Invention

The present invention is directed to an adjustable acupressure device structured to permit a user to accurately position a pressure assembly such that a pressure applicator overlies a specific pressure point on his or her body and, thereafter, apply a measured amount of pressure to the specific pressure point via the pressure applicator.

2. Description of the Related Art

Eastern medicine has made use of various acupuncture and acupressure techniques for hundreds if not thousands of years, and these techniques have been shown to alleviate a wide variety of ailments. A key to these types of treatment techniques is locating a proper point or points on a person's body to insert a needle or to apply pressure. In the case of acupressure, the actual amount of pressure applied, once the correct point has been located, is also a primary factor in the effectiveness of the treatment.

As such, devices have been developed to apply some amount of pressure to some portion of a person's body in hopes of achieving some form of relief for the user. Among these devices is an inflatable headband mechanisms which, when inflated, applies pressure about the users head. Other devices include various adjustable straps having either fixed or adjustable pressure application portions which may be affixed to various portions of the user's body, once again, attempting to impart the benefits of accurately placed pressure onto the user's body. Among the shortcomings of such devices is the inability to assure specific placement of a pressure application portion to a specific point on the user's body as required to achieve the desired therapeutic benefits of the treatment technique. Additionally, many of these devices fail to permit repetitive application of a relatively fixed amount of pressure, even assuming the proper pressure application point on the body were located. Another common drawback to such devices is that they tend to be uncomfortable and awkward for the user to wear for prolonged periods, and as such, many user's simply fail to take advantage of the potential benefits of acupressure treatment techniques.

Among the most common maladies reported to physicians today is a headache. Research has shown that when properly applied, acupressure may serve to relieve the severity or intensity of headaches, even migraine headaches. Furthermore, acupressure techniques have been shown to significantly reduce the frequency of occurrences of headaches when properly applied.

In particular, application of a proper amount of pressure for a specified period of time to the LI-4 or “hegu” point of the body has been found to significantly reduce and/or eliminate the pain and discomfort of a headache or migraine in many people. A hegu point is generally located in the fleshy part of each hand between the thumb and forefinger. More precisely, the hegu point is located between the first and second metacarpals of a person's hand.

One device, in the form of a glove, has been developed in an attempt to apply pressure to the hegu point in order to impart the therapeutic properties of acupressure to user's that may apply the device themselves. Once again, however, this device suffers the same shortcomings discussed above with regard to other acupressure devices. To begin, the device includes a bladder attached to the glove, the bladder being easily shifted about in the general area of a user's thumb and forefinger, so once again, accurate placement of a pressure application portion to overly the hegu is difficult, especially for an average user having little to no medical training or experience. Also, this device includes a squeeze bulb attached via an air hose to the inflatable bladder, the inflatable bladder being filled by the user without means to determine whether the correct amount of pressure is being applied, often resulting in either too little or too much pressure, either of which may reduce or eliminate any benefit which may otherwise be achieved. Additionally, when the device is installed and inflated, the hose and bulb remain attached and make it awkward for the user to go about their normal daily routine while utilizing the device.

Thus, it would be beneficial to provide an acupressure device including means to permit an average user, i.e., a user without medical training and/or experience, to accurately position the device on his or her body so as to permit an application of pressure to a specific portion of the user's body. It would further be helpful for such an acupressure device to include means to allow a user to accurately control an amount of pressure applied to a specific portion of the user's body. Also, it would be preferable to provide an acupressure device which may be worn by a user in an unobtrusive manner such that the user may go about his or her normal daily routine while the device is in position and applying a controlled amount of pressure to a specific portion of the user's body.

### SUMMARY OF THE INVENTION

As noted above, the present invention is directed to an adjustable acupressure device structured to apply pressure to a portion of a user's body. The device comprises an attachment assembly structured to facilitate disposition of the device on the user's body and, in at least one embodiment, the attachment assembly permits removable positioning of the device on the user's body. The attachment assembly includes at least one placement indicator which is structured to facilitate positioning the device proximate the portion of the user's body to which pressure is to be applied. In one further embodiment, the attachment assembly may comprise a plurality of placement indicators, the placement indicators may include tick marks, letters, numbers, etc., or a combination of the foregoing, to allow the average user with little to no medical education and/or experience to properly position the device on the user's body.

The adjustable acupressure device of the present invention also includes a pressure assembly which is interconnected to the attachment assembly, the pressure assembly having a pressure applicator connected thereto. The pressure assembly is structured to overly the portion of the user's body on which pressure is to be applied such that the pressure applicator contacts a specific portion of the user's body. In one embodiment, the adjustable acupressure device may be utilized to apply pressure to a user's hegu point, that being the point located between the user's thumb and forefinger, as described in further detail below, in order to alleviate and/or eliminate headache pain.

In at least one embodiment, the pressure assembly of the inventive device comprises an inflatable bladder, the inflatable bladder having an expandable portion and a non-expandable portion. The pressure applicator is connected to the expandable portion of the inflatable bladder to permit the application of a preselected amount of pressure to the specific portion of the user's body.

At least one embodiment of the adjustable acupressure device of the present invention includes an inflation mechanism and a pressure gauge, each being interconnected to the pressure assembly and structured to facilitate the application of the preselected amount of pressure to the specific portion of the user's body. One further embodiment of the present invention comprises an inflation mechanism removably interconnected to the pressure assembly, thereby allowing the user to remove the inflation mechanism once the adjustable acupressure device is in position on a portion of the user's body and the inflatable bladder is inflated to a predetermined pressure, thereby permitting the user to go about their normal daily routine unencumbered by the device, yet obtaining the benefits provided thereby.

These and other objects, features and advantages of the present invention will become more clear when the drawings as well as the detailed description are taken into consideration.

### BRIEF DESCRIPTION OF THE DRAWINGS

For a fuller understanding of the nature of the present invention, reference should be had to the following detailed description taken in connection with the accompanying drawings in which:

FIG. 1 is a perspective view of an adjustable acupressure device in accordance with the present invention disposed in an operative position on a user's hand.

FIG. 2 is an elevation of one preferred embodiment of a pressure assembly of the present invention illustrating an inflatable bladder disposed in an inflated configuration.

FIG. 3 is a partial plan view of the adjustable acupressure device in accordance with the embodiment of FIG. 1.

FIG. 4 is a cross-sectional elevation of the pressure assembly of FIG. 2 illustrated the inflatable bladder disposed in a deflated configuration.

Like reference numerals refer to like parts throughout the several views of the drawings.

### DETAILED DESCRIPTION OF THE PREFERRED EMBODIMENT

The present invention is directed to an adjustable acupressure device, generally as shown at 10 throughout the figures. The adjustable acupressure device 10 is structured to apply an amount of pressure to a portion of a user's body. In at least one embodiment, the portion of the user's body to which pressure is to be applied, is proximate the user's hands and, more specifically, in one preferred embodiment, the adjustable acupressure device 10 of the present invention is utilized to apply an amount of pressure to a hegu point of the user's hand, as discussed in more detail below.

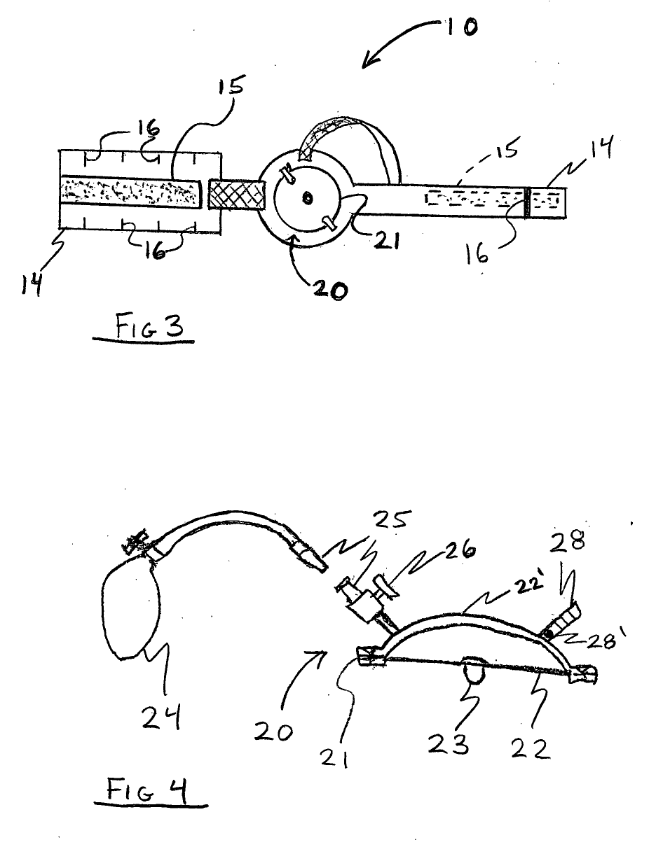

To begin, the adjustable acupressure device 10 of the present invention comprises an attachment assembly 12. The attachment assembly 12 is structured to facilitate removably positioning the adjustable acupressure device 10 on the user's body, and comprises at least one adjustable attachment member 14. The adjustable attachment member 14 preferably comprises an elongated configuration, as shown in the figures, structured to encircle at least a portion of the user's body, thereby permitting secure, yet removable positioning of the adjustable acupressure device 10 in such a manner that the device 10 will not move or shift about the user's body. In one preferred embodiment, the attachment assembly 12 comprises a plurality of adjustable attachment members 14, each comprising an elongated configuration, as illustrated in FIG. 3.

In addition, at least one attachment member 14 comprises an attachment mechanism, as at 15, structured to facilitate adjustable interconnection of the adjustable attachment members 14 to one another thereby permitting removable positioning of the adjustable acupressure device 10 on the portion of the user's body. As illustrated in the figures, and as best shown in FIG. 3, the attachment mechanism 15 in one preferred embodiment comprises a hook and loop type of fastener being affixed to each of the plurality of adjustable attachment members 14. As will be appreciated, the attachment mechanism 15 permits the user to adjust at least one overall dimension of the attachment assembly 12, for example, a circumference being at least partially defined by the adjustable attachment members 14 utilized to encircle a portion of the users body. Thus, the positioning of the adjustable acupressure device 10 on the user's body may be adjusted by simply varying an amount of overlap between the adjustable attachment members 14. It is, however, understood to be within the scope and intent of the present invention for the attachment mechanism 15 to comprise any type of fastening mechanism which permits the adjustable interconnection of the plurality of attachment members 14 to one another, including but not limited to, snaps, buttons, clips, straps, clasps, etc.

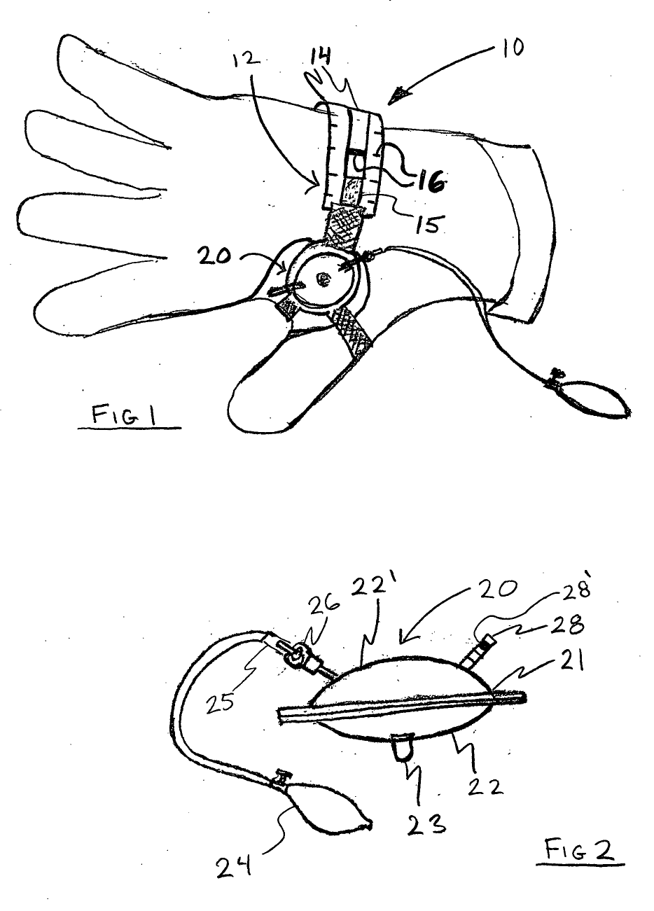

The attachment assembly 12 of the present invention further comprises at least one placement indicator 16 disposed so as to allow visual observation of the indicator 16 when the adjustable acupressure device 10 is removably positioned on a portion of the user's body. More in particular, the placement indicator 16 is structured to facilitate accurate positioning of the device 10 proximate the portion of the user's body to which pressure is to be applied via visual alignment of the placement indicator 16, as discussed below. The attachment assembly 12, in one preferred embodiment, comprises a plurality of placement indicators 16, as are illustrated in FIGS. 1 and 3. The placement indicators 16 of the present-invention may include, but are not limited to, tick marks, letters, numbers, etc., or a combination of the foregoing, as noted above. Additionally, one or more of the placement indicators 16 may be color coded to further aid visual observation by the user.

In a preferred embodiment of the present invention, each adjustable attachment member 14 comprises at least one placement indicator 16 disposed such that visual alignment of the placement indicators 16 relative to one another is indicative of a preferred and accurate positioning of the adjustable acupressure device 10 on a portion of the user's body, such visual alignment being readily accomplished by simply varying the amount of overlap between the adjustable attachment members 14, as described above.

Specifically, the size and proportions of different user's bodies are widely varied, such that the amount of overlap of the adjustable attachment members 14 required to accurately position the adjustable acupressure device 10 on the same portion of the body of different user's will also vary widely. Thus, the plurality of placement indicators 16 allows an average user, i.e., a user without medical training and/or experience, to accurately position the adjustable acupressure device 10 on his or her body so as to permit the application of pressure to a specific portion of the user's body, by simple visual alignment of the plurality of placement indicators 16 in a predetermined alignment with one another. It is understood that trained personnel may be required to assist a user to initially identify a “predetermined alignment” of the placement indictors 16 which results in the accurate positioning of the adjustable acupressure device 10 on the portion of the user's body to which pressure is to be applied, after which, however, the user may simply visually align the placement indicators 16 in the “predetermined alignment” to assure accurate positioning of the device 10 on the portion of his or her body.

As an example, in one preferred embodiment, the adjustable acupressure device 10 of the present invention is utilized to apply an amount of pressure to a user's hegu point. The hegu point is located in the fleshy portion of the hand between the thumb and forefinger, or, more specifically, and as noted above, between the first and second metacarpals. Also as previously indicated, proper and repetitive application of pressure to a user's hegu point has been shown to reduce and/or eliminate headache pain. Thus, the present invention facilitates the proper application of pressure to a user's hegu point by allowing accurate and repetitive placement of a pressure applicator 23, discussed more fully below, in contact with the user's hegu point when the adjustable acupressure device 10 is disposed in an operative position on the user's hand, the operative position being at least partially defined by the plurality of placement indicators 16 being disposed in a predetermined alignment while the device 10 is removably positioned on the user's hand.

The adjustable acupressure device 10 of the present invention also comprises a pressure assembly, generally as shown at 20 throughout the figures. The pressure assembly 20 is interconnected to the attachment assembly 12 and, in a preferred embodiment, the pressure assembly 20 is securely interconnected to the attachment assembly 12 such that movement or displacement of the pressure assembly 20 relative to the attachment assembly 12 is minimal, particularly when the adjustable acupressure device 10 is removably positioned on a portion of the user's body. Thus, the pressure assembly 20 is structured to remain substantially in position relative to a portion of a user's body when the adjustable acupressure device 10 of the present invention is securely yet removably positioned on the user via the attachment assembly 12.

The pressure assembly 20 comprises a pressure applicator 23, as noted above, the pressure applicator 23 being structured to contact a portion of the user's body when disposed in an operative position. More specifically, the pressure applicator 23 preferably comprises a rigid elongated projection extending outwardly from the pressure assembly 20 and directed towards the user's body such that the pressure applicator 23 contacts a specific portion of the user's body when the adjustable acupressure device 10 is removably positioned on the user. As discussed in greater detail below, the pressure applicator 23 is structured to apply a preselected amount of pressure to the specific portion of the user's body. As noted above, in one preferred embodiment, the specific portion of the user's body is the hegu point located in the fleshy portion of the hand between the thumb and forefinger.

To facilitate the application of a preselected amount of pressure to a portion of the user's body, the pressure assembly 20 further comprises an inflatable bladder 21. The inflatable bladder 21 includes an expandable portion 22 structured to expand and contract upon application and release of pressure thereto, respectively, and may be comprises of any of a number of flexible, pliable materials that are substantially impervious to the transfer of air or other compressible fluids therethrough. In a preferred embodiment, the inflatable bladder 21 further comprises a non-expandable portion 22′, such as is illustrated in FIGS. 2 and 4. The non-expandable portion 22′ preferably comprises one or more rigid, inflexible materials which are also substantially impervious to the transfer of air or other compressible fluids therethrough.

The expandable portion 22 is preferably disposed opposite and spaced apart from the non-expandable portion 22′, also as illustrated in FIGS. 2 and 4. More in particular, the expandable portion 22 is preferably disposed adjacent to and in an at least partially contacting relationship with the user's body when the adjustable acupressure device 10 is removably positioned on the user's body, while the non-expandable portion 22′, being oppositely disposed, remains in a substantially non-contacting relationship with the user's body. The pressure applicator 23 is preferably connected to the expandable portion 22 of the inflatable bladder 21, to facilitate contact between the pressure applicator 23 and a specific portion of the user's body to which pressure is to be applied, such as, by way of example only, the user's hegu point, and in one preferred embodiment, the pressure applicator 23 is disposed in direct contact with a specific portion of the user's body.

In one further preferred embodiment of the present invention, the inflatable bladder 21, and more specifically, the expandable portion 22 and the non-expandable portion 22′, comprise a material of construction that is light transmissive. As utilized herein, light transmissive shall be defined as a material which permits visual observation therethrough by the naked eye under normal, ambient lighting conditions, i.e. natural daylight or ordinary home or office lighting. The light transmissive construction of the inflatable bladder 21 serves to further facilitate accurate positioning of the adjustable acupressure device 10 of the present invention on a portion of the user's body. More in particular, the light transmissive inflatable bladder 21 permits a user to visually observe a point of contact between the pressure applicator 23 and the user's body and to adjust the positioning of the adjustable acupressure device 10 on the user's body as may be necessary to position the pressure applicator 23 into direct contact with a specific point of the user's body, such as the hegu point.

The pressure assembly 20 of the present invention also comprises an inflation mechanism 24 disposed in an operative association therewith, the operative association being at least partially defined by a fluid transferring relationship between the inflation mechanism 24 and the inflatable bladder 21 to permit inflation of the inflatable bladder 21 to at least one predetermined pressure. As illustrated in the figures, the inflation mechanism 24 may comprise a squeeze bulb interconnected to the inflatable bladder 21 by a length of flexible tubing, wherein squeezing the bulb forces air from the squeeze bulb, under pressure, into the inflatable bladder 21 through the flexible tubing. As will be appreciated, the squeeze bulb of the inflation mechanism 24 of this embodiment may be squeezed repeatedly to force more air, under pressure, into the inflatable bladder, until a desired predetermined pressure is attained in the inflatable bladder 21. As will further be appreciated, the inflation mechanism 24 of the present invention may comprise any number of devices structured to deliver air, or other compressible matter, into the inflatable bladder 21 to attain the desired predetermined pressure therein including, but not limited to, a compressed gas delivery system, a hand operated piston pump, an electrical pump, a bellows, etc., just to name a few.

In a further preferred embodiment of the present invention, a quick connect fitting 25, as are readily available, is employed to facilitate disposition of the inflation mechanism 24 into operative association with the inflatable bladder 21. Specifically, the quick connect fitting 25 is structured to permit quick, easy, and removable connection of the inflation mechanism 24, such as one end of the flexible tubing, to the inflatable bladder 21, preferably, the non-expandable portion 22′ of the inflatable bladder 21, in a fluid transferring relationship, as illustrated in FIG. 2. The opposite end of the flexible tubing is connected to the squeeze bulb or other delivery device, such that compressed air, or another compressed fluid, can be transferred into the inflatable bladder 21, under pressure, from the delivery device. The quick connect fitting 25 is further, and perhaps, more importantly, structured to permit the flexible tubing and the delivery device of the inflation mechanism 24 to be quickly and easily disconnected from the inflatable bladder 21, once the adjustable acupressure device 10 has been removably positioned on a portion of the user's body, and the inflatable bladder 21 has been inflated to a predetermined pressure. Thus, the quick connect fitting 25 permits the adjustable acupressure device 10 of the present invention to be worn by a user in an unobtrusive manner, i.e., without the inflation mechanism 24 attached thereto, such that the user may go about his or her normal daily routine while the device 10 is in position and applying a preselected amount of pressure to a specific portion of the user's body.

The pressure assembly 20 also further comprises a pressure release valve 26. As illustrated in the figures, the pressure release valve 26 is preferably disposed between the inflatable bladder 21 and the quick connect fitting 25. In the preferred illustrated embodiment of the present invention, the pressure release valve 26 serve dual purposes. First, the pressure release valve 26, as the name implies, may be operated to release pressure from within the inflatable bladder 21, once the user has completed the application of an amount of pressure to a portion of the body for a period of time. In addition, the pressure release valve 26 includes a backflow prevention mechanism, such as a check valve, to permit an amount of compressed air, or other compressible fluid, to be retained within the inflatable bladder 21 and substantially maintain the amount of pressure applied to the portion of the user's body for the period of time necessary for a particular treatment cycle, until the user elects to deflate the bladder 21 via operation of the pressure release valve 26.

An additional feature of the pressure assembly 20 of the present invention is the incorporation of a pressure gauge 28. As illustrated in the figures, and similar to the pressure release valve 26, the pressure gauge 28 is preferably disposed in fluid communication with the non-expandable portion 22′ of the inflatable bladder 21, however, it is understood to be within the scope and intent of the present invention for the pressure gauge 28 to be disposed in fluid communication with the expandable portion 22. The pressure gauge 28 of the present invention is structured to provide a visual indication of at least one predetermined pressure within the inflatable bladder 21. In a preferred embodiment, the pressure gauge 28 is structured to provide a visual indication of each of a plurality of predetermined pressures within the inflatable bladder 21, to permit the user to selectively an appropriate predetermined amount of pressure with which to inflate the inflatable bladder 21. As indicated above with respect to the initial identification of the “predetermined alignment” of the placement indictors 16, trained personnel may be required to assist the user to establish which of the plurality of “predetermined pressures” is appropriate, after which, however, the user can quickly and easily apply the correct predetermined amount of pressure to a portion of his or her body by simply inflating the inflatable bladder 21 to the appropriate “predetermined pressure” through visual observation of the pressure gauge 28. Each “predetermined pressure” within the inflatable bladder 21 further corresponds to a preselected amount of pressure being applied to a portion of the user's body.

More in particular, if the pressure gauge 28 indicates that more pressure is required, the user simply squeezes the bulb one or more times, or operates an alternate delivery device until the desired predetermined pressure is attained, as indicated by the pressure gauge 28.. Alternatively, if too much pressure is present, the user may simple operate the pressure release valve 26 to release pressure from within the inflatable bladder 21, until the desired predetermined amount of pressure is indicated by the pressure gauge 28. As illustrated in the figures, the pressure gauge 28 comprises an elongated tube having a series of visible tick marks, and a spherical pressure indicator 28′ which moves within the tube to indicate the various pressures indicated by the tick marks, as illustrated by comparison of FIGS. 2 and 4. In at least one embodiment, the tick marks of the pressure gauge 28 are structured to indicate a “High”, “Medium”, and “Low” predetermined pressure within the inflatable bladder 21. For example, FIG. 2 illustrates the pressure indicator 28′ in an elevated position near the top of tube while the inflatable bladder 21 is disposed in an inflated configuration, indicative of a “High” predetermined pressure within the inflatable bladder 21, whereas FIG. 4 shows the pressure indicator 28′ positioned near the bottom of the tube, indicative of a deflated, i.e., substantially non-pressurized, configuration of the inflatable bladder 21. Of course, it is within the scope and intent of the present invention for alternate types of pressure gauges 28 and/or pressure indicators 28′ to be utilized such as, by way of example only, diaphragm gauges having needle or digital LED indicators which may indicate a pressure in pounds per square inch, etc.

Lastly, and looking once again to FIGS. 2 and 4 which illustrate the inflatable bladder 21 in an inflated configuration and a deflated configuration, respectively, the importance of the non-inflatable portion 22′ of the inflatable bladder 21 will become apparent. Specifically, the non-inflatable portion 22′ serves to direct a predetermined pressure within the inflatable bladder 21 to the expandable portion 22, such that a preselected amount of pressure may be applied to a specific portion of the user's body by the pressure applicator 23. If the entire inflatable bladder 21 were comprised of expandable material, a significantly greater amount of compressed air or other compressible material would be required to achieve the same predetermined pressure within the inflatable bladder 21, as the upper portion would be free to expand relatively unrestricted against atmospheric pressure.

Since many modifications, variations and changes in detail can be made to the described preferred embodiment of the invention, it is intended that all matters in the foregoing description and shown in the accompanying drawings be interpreted as illustrative and not in a limiting sense. Thus, the scope of the invention should be determined by the appended claims and their legal equivalents.

Now that the invention has been described,

What is claimed is:

1. An adjustable acupressure device structured to apply pressure to a portion of a user's body, said device comprising:
an attachment assembly structured to removably position said device on the user's body,
a pressure assembly interconnected to said attachment assembly and comprising a pressure applicator structured to contact the portion of the user's body when disposed in an operative position,
said attachment assembly having at least one placement indicator structured to facilitate disposition of said pressure applicator in said operative position, and
said pressure applicator structured to apply a preselected amount of pressure to the portion of the user's body.

2. The device as recited in claim 1 wherein said pressure assembly comprises an inflatable bladder having an expandable portion.

3. The device as recited in claim 2 wherein said inflatable bladder further comprises a non-expandable portion comprising a substantially rigid material.

4. The device as recited in claim 3 wherein said inflatable bladder is structured such that said expandable portion is disposed in an opposing and spaced apart relation to said non-expandable portion.

5. The device as recited in claim 4 wherein said pressure applicator is connected to said expandable portion of said inflatable bladder.

6. The device as recited in claim 2 wherein said inflatable bladder comprises a light transmissive material to further facilitate disposition of said pressure applicator in said operative position.

7. The device as recited in claim 2 wherein said pressure assembly further comprises an inflation mechanism disposed in an operative association with said inflatable bladder and structured to permit inflation of said inflatable bladder to a predetermined pressure.

8. The device as recited in claim 7 wherein said pressure applicator is structured to apply said preselected amount of pressure to the portion of the user's body upon inflation of said inflatable bladder to said predetermined pressure.

9. The device as recited in claim 2 wherein said pressure applicator is further structured to apply said preselected amount of pressure to the portion of the user's body while said inflatable bladder is inflated to a predetermined pressure.

10. An adjustable acupressure device structured to apply pressure to a portion of a user's body, said device comprising:
an attachment assembly structured to facilitate disposition of said device on the user's body,
said attachment assembly comprising at least one placement indicator structured to facilitate positioning said device proximate the portion of the user's body,
a pressure assembly interconnected to said attachment assembly having a pressure applicator structured to overly the portion of the user's body,
said pressure assembly comprises an inflatable bladder having an expandable portion,
said pressure applicator being connected to said expandable portion of said inflatable bladder,
said pressure applicator structured to apply a preselected amount of pressure to the portion of the user's body, and
said pressure assembly comprising a pressure gauge to facilitate application of said preselected amount of pressure to the portion of the user's body.

11. The device as recited in claim 10 wherein said pressure assembly further comprises an inflation mechanism disposed in an operative association therewith, said inflation mechanism structured to inflate said inflatable bladder to a predetermined pressure.

12. The device as recited in claim 10 wherein said pressure gauge is structured to indicate at least one predetermined pressure of said inflatable bladder.

13. The device as recited in claim 10 wherein said pressure gauge is structured to indicate each of a plurality of predetermined pressures of said inflatable bladder.

14. The device as recited in claim 10 wherein said attachment assembly comprises a plurality of placement indicators structured to facilitate positioning said device proximate the portion of the user's body.

15. The device as recited in claim 10 wherein said attachment assembly comprises a plurality of placement indicators structured to facilitate positioning said pressure applicator in an operative position on the user's body, said operative position being at least partially defined by said pressure applicator disposed in contact with the portion of the user's body.

16. The device as recited in claim 15 wherein said operative position is further defined by said pressure applicator disposed in contact a specific portion of the user's body.

17. The device as recited in claim 16 wherein the specific portion of the user's body is a hegu point located between a user's thumb and forefinger.

18. An adjustable acupressure device structured to apply pressure to a portion of a user's body, said device comprising:
an attachment assembly structured to securely yet removably position said device on the user's body,
a pressure assembly interconnected to said attachment assembly and comprising a pressure applicator structured to contact the portion of the user's body when disposed in an operative position,
said attachment assembly having a plurality of placement indicators to facilitate disposition of said pressure applicator in said operative position,
said pressure assembly comprising an inflatable bladder having an expandable portion and a non-expandable portion comprising a substantially rigid material,
an inflation mechanism disposed in an operative association with said inflatable bladder and structured to permit inflation of said inflatable bladder to a predetermined pressure, and
said pressure applicator being connected to said expandable portion of said inflatable bladder and structured to apply a preselected amount of pressure to the portion of the user's body upon inflation of said inflatable bladder to said predetermined pressure.

19. The device as recited in claim 18 further comprising a pressure gauge structured to indicate at least one predetermined pressure of said inflatable bladder.

20. The device as recited in claim 18 further comprising a pressure gauge structured to indicate a plurality of predetermined pressures of said inflatable bladder.

21. The device as recited in claim 18 wherein said inflation mechanism is removably connected to said inflatable bladder.

22. The device as recited in claim 21 further comprising a quick connect fitting structured to facilitate said removable connection of said inflation mechanism to said inflatable bladder.

23. The device as recited in claim 22 wherein said quick connect fitting is further structured to permit disconnection of said inflation mechanism from said inflatable bladder while retaining said predetermined pressure within said inflatable bladder.

24. The device as recited in claim 18 wherein said pressure assembly further comprises a pressure release valve structured to permit deflation of said inflatable bladder.
